(19) United States
(12) Patent Application Publication
(10) Pub. No.: US 2024/0173068 A1
(43) Pub. Date: May 30, 2024

(54) SURGICAL END EFFECTOR DETECTION

(71) Applicant: GYRUS MEDICAL LIMITED, Cardiff (GB)

(72) Inventor: Matthew RUNNALLS, Cardiff (GB)

(73) Assignee: GYRUS MEDICAL LIMITED, Cardiff (GB)

(21) Appl. No.: 18/521,275

(22) Filed: Nov. 28, 2023

Related U.S. Application Data
(60) Provisional application No. 63/428,915, filed on Nov. 30, 2022.

(51) Int. Cl. A61B 18/14

(52) U.S. Cl. CPC A61B 18/148; A61B 2018/00601; A61B 2018/1495

(57) ABSTRACT

A surgical instrument including a means for determining the presence of an end effector and determining the type of end effector which is coupled to the surgical instrument handpiece. The end effector includes a ring-shaped magnet, and the handpiece includes a hall-effect sensor for determining the presence and magnitude of the magnetic field produced by the ring-shaped magnet. This allows the end effector to be characterised even where the end effector is rotatably coupled to the handpiece of the surgical instrument.

### TECHNICAL FIELD

Embodiments of the present invention described herein relate to a surgical end effector including a magnet to allow the detection of the coupling of the end effector with a handle portion of an electrosurgical instrument.

### BACKGROUND TO THE INVENTION

Surgical instruments, and in particular electrosurgical instruments, have a wide variety of uses, for example mechanical cutting, ablation or coagulation during surgery. Electrosurgical instruments may therefore be compatible with a number of different electrosurgical end effectors, allowing them to provide multiple functions. In some cases, the electrosurgical end effector may rotate during use, for example to provide a mechanical cutting function.

There is a need to provide a system which can recognise the presence or connection of an electrosurgical end effector and identify the type of electrosurgical end effector connected.

### SUMMARY OF THE INVENTION

Embodiments of the present invention provide an improved electrosurgical end effector. The electrosurgical end effector may be coupled to a surgical instrument handpiece, and the electrosurgical end effector comprises means which allow the presence of the electrosurgical end effector and the type of electrosurgical end effector to be determined. For example, the electrosurgical end effector includes a ring-shaped magnet which may be detected by a hall-effect sensor included in the surgical instrument handpiece. Variations in the strength of the magnetic field detected, for example caused by the strength of the magnet or the distance between the magnet and the hall-effect sensor, may allow different types of end effector to be distinguished. This simplifies the operation of the system, and may allow the surgical instrument to automatically modify its mode of operation in dependence on the type of detected end effector. The use of a ring-shaped magnet beneficially allows a rotatable end effector to be inserted into the hand-piece in any rotation and still be detected.

In view of the above, in a first aspect of the present disclosure, an electrosurgical end effector for rotatably coupling to a surgical instrument handpiece, the surgical instrument handpiece comprising a hall effect sensor, the electrosurgical end effector comprising: a distal end; a proximal end comprising: a ring-shaped magnet; a connection means configured for rotatably coupling to the surgical instrument handpiece.

The ring-shaped magnet may be a face-to-face polarised magnet. A position of the magnet on the proximal end of the electrosurgical end effector may identify a function of the electrosurgical end effector. The position of the magnet on the proximal end of the electrosurgical end effector may be such that when the end effector is coupled to the surgical instrument handpiece, the relative position of the magnet and the hall effect sensor identifies the function of the electrosurgical end effector. The position of the magnet along a rotational axis of the electrosurgical end effector identifies the function of the electrosurgical end effector.

The ring-shaped magnet may be a radially polarised magnet. The polarisation of the radially polarised magnet may identify a function of the electrosurgical end effector.

The electrosurgical end effector may be an RF electrosurgical end effector, and the distal end of the electrosurgical end effector may comprise an active electrode and a return electrode.

The electrosurgical end effector may be a rotary shaver end effector, and the distal end of the electrosurgical end effector comprises a rotary shaver arrangement.

According to a second aspect of the invention, an electrosurgical instrument may comprise: a handpiece; and an electrosurgical end effector. The handpiece may comprise a hall-effect sensor. The ring-shaped magnet of the electrosurgical end effector may be aligned with the hall-effect sensor. The ring-shaped magnet of the electrosurgical end effector is offset with respect to the hall-effect sensor along a rotational axis of the electrosurgical end effector. The hall-effect sensor may be configured to detect a magnetic field produced by the ring-shaped magnet.

The electrosurgical instrument may further comprise a processor configured to determine a function of the electrosurgical end effector based on an output of the hall-effect sensor.

According to a third aspect of the invention, a method of detecting the coupling of an electrosurgical end effector comprising a ring-shaped magnet to a surgical instrument handpiece, the method comprising: detecting, using a hall-effect sensor, the presence of a magnetic field produced by the ring-shaped magnet.

The method may further comprise determining a function of the electrosurgical end effector based on a magnitude of the detected magnetic field. The method may further comprise supplying a control signal to the electrosurgical end effector in dependence on the determined function of the electrosurgical end effector.

The ring-shaped magnet may be a face-to-face polarised magnet, and wherein: determining the function of the electrosurgical end effector further comprises determining an offset between the ring-shaped magnet and the hall-effect sensor along a rotational axis of the electrosurgical end effector.

The ring-shaped magnet may be a radially polarised magnet, and wherein: determining the function of the electrosurgical end effector further comprises determining the polarisation of the radially polarised magnet.

### BRIEF DESCRIPTION OF THE DRAWINGS

Embodiments of the invention will now be described by way of example only and with reference to the accompanying drawings, wherein like reference numerals refer to like parts, and wherein:

FIG. 1 is a side view of an electrosurgical instrument including an electrosurgical end effector and a handle portion;

FIG. 2 is a view of an electrosurgical instrument coupled to an electrosurgical generator;

FIG. 3 is a cutaway view of the connection of the electrosurgical end effector and the handle portion of FIG. 1;

FIG. 4a is a first simplified view of a face-to-face polarised magnet offset from a hall-effect sensor;

FIG. 4b is a second simplified view of a face-to-face polarised magnet offset from a hall-effect sensor;

FIG. 4c is a third simplified view of a face-to-face polarised magnet offset from a hall-effect sensor;

FIG. 5a is a first simplified view of a radially polarised magnet;

FIG. 5b is a second simplified view of a radially polarised magnet;

FIG. 6 is a flowchart of a method for determining the presence of a surgical end effector.

### DESCRIPTION

End effectors may, in use, be inserted into a patient to allow a surgical operation to be performed. A number of different end effectors may be coupled to a surgical instrument, allowing the surgical instrument to provide a variety of functions. Further, the end effector may be removed and replaced following surgery, allowing easy sterilisation. By providing a magnet on the end effector and a hall-effect sensor on the handle portion of the surgical instrument, the presence of the end effector may be registered, providing the surgical instrument with confirmation that an end effector is coupled to the handled portion.

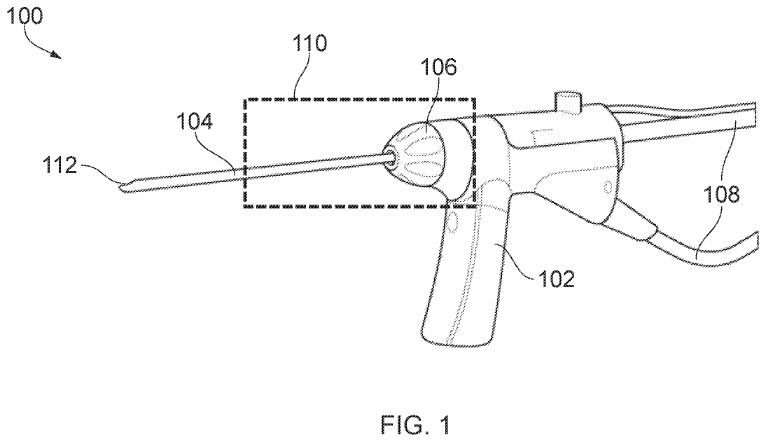

FIG. 1 shows an electrosurgical instrument 100. The instrument 100 includes a proximal handle portion or surgical instrument handpiece 102 and an end effector assembly 104 coupled to the handle portion 102 at a connection portion 106. The end effector assembly 104 extends in a distal direction away from the proximal handle portion 102. The end effector assembly 104 is removably or detachably coupled to the handle portion 102 at the connection portion 106.

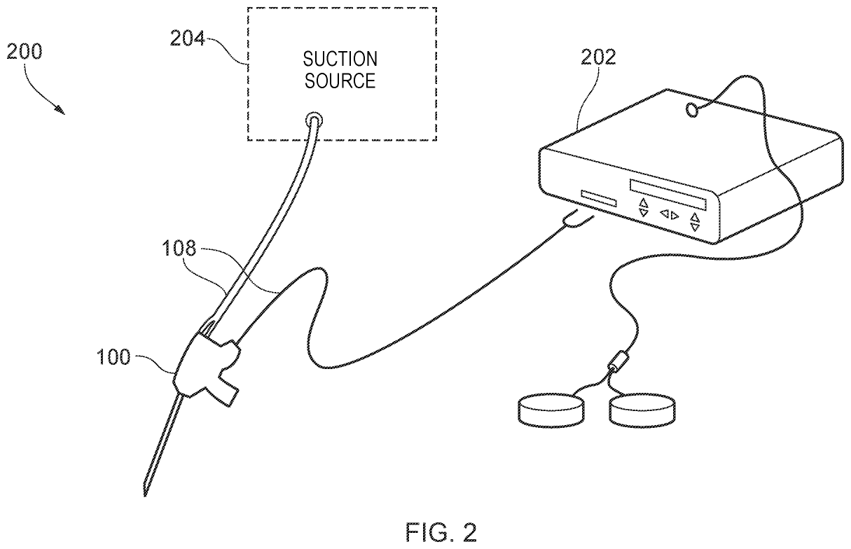

The electrosurgical instrument 100 may include a number of connection cords or tubes 108, for connecting the electrosurgical instrument 100 to an electrosurgical generator. For example, FIG. 2 shows an electrosurgical system 200 comprising the electrosurgical instrument 100 coupled to an electrosurgical generator 202 and a suction source 204 via the connection cords or tubes 108. The RF generator 202 may supply an RF electrosurgical signal to the electrosurgical instrument 100. The tubes 108, or in some cases a single tube 108, may connect the electrosurgical instrument to an irrigation and suction source 204 to provide irrigation and suction.

The electrosurgical instrument 100 may be compatible with a number of different types of electrosurgical end effector 104. For example, an end effector 104 which provides a mechanical cutting function, sometimes referred to as a shaver, an end effector which provides an RF coagulation and/or ablation function, or a combined RF shaver.

The removable end effector 104 may be removed following a surgical operation and discarded, whilst the handle portion 102 may be reused following sterilisation. The end effector 104 is more likely to become contaminated during use. Allowing easy removal of the end effector allows the handle portion 102 to be more easily sterilised.

Electrosurgical end effectors 104 may rotate during use or operation. For example, an RF shaver end effector 104 may continuously rotate relative the handpiece 102 to provide a cutting function. Alternatively, the end effector 104 may be selectively or controllably rotatable relative to the handpiece 102 so as to allow an active edge 112 at the distal end of the end effector 104 to be rotated into a selected position. The active edge 112 may include, for example, RF electrodes.

As the end effector 104 may be removed and replaced, a system is needed to identify the presence of the end effector 104 and to further determine the type of end effector 104 coupled to the handle portion 102. For example, a magnet may be included in the end effector 104 and a hall-effect sensor in the connection portion 106 or the handle portion 102 of the electrosurgical instrument 100. The hall effect sensor may detect the presence of the magnet which, as the magnet is part of the end effector 104, also results in the detection of the end effector 104.

However, where the end effector 104 is rotatably coupled to the handle portion 102, the end effector 104 may be inserted or attached to the handle portion 102 in any orientation or rotational position. As the end effector 104 may be inserted in any rotational position, the magnet may also be inserted in any rotation, and thus not align with the hall effect sensor in the handpiece. This may result in the system not detecting the presence of the end effector 104, or mis-identifying the end-effector 104.

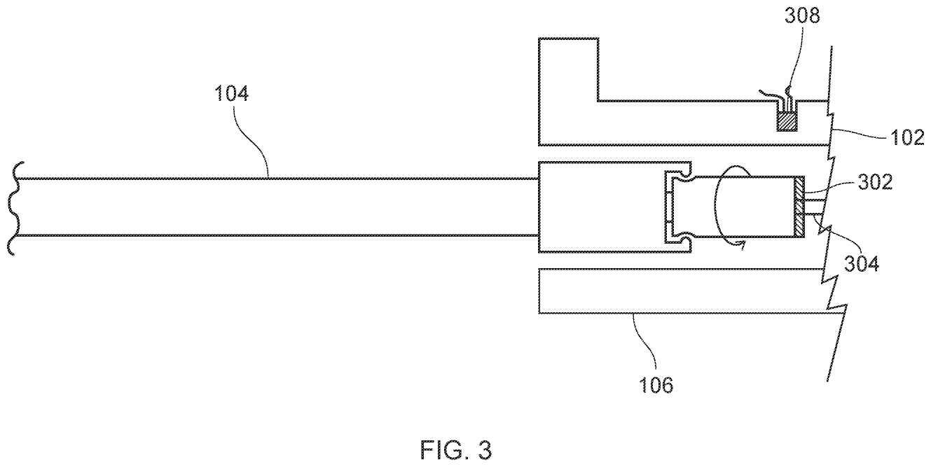

FIG. 3 shows a cutaway view of the region 110 of the electrosurgical instrument 100 of FIG. 1 including an electrosurgical end effector 104 coupled to the handle portion 102 of the electrosurgical instrument 100 at the connection portion 106. The proximal end of the electrosurgical end effector 104 comprises a connection means 304 for coupling to the connection portion 106 of the electrosurgical instrument 100. The electrosurgical end effector 104 comprises a magnet 302 near the proximal end of the end effector 104 (e.g. at or near to the end which is coupled to the handle portion 102). The connection portion 106 comprises a hall effect-sensor 308. The magnet 302 is a ring magnet, or ring-shaped magnet, located on the electrosurgical end effector 104 such that in use, when the electrosurgical end effector 104 is coupled to the handle portion 102, the hall-effect sensor 308 can detect the presence of the magnet 302 and the magnetic field produced by the magnet.

The coupling between the electrosurgical end effector 104 and the handle portion 102 at the connection portion 106 is a rotatable coupling, such that, in use, the electrosurgical end effector 104, or part of the electrosurgical end effect 104, may rotate. The rotation may be along the axis of the electrosurgical end effector running from the handle portion 102 to the active edge 112 of the end effector 104. As the coupling is rotatable, the electrosurgical end effector 104 may be inserted into the coupling portion 104 in any rotational position.

The hall-effect sensor 308 detects the presence of the magnetic field produced by the ring-shape magnet, and therefore detects the presence of the end effector 104 to which the magnet 302 is attached. By using a ring-shaped magnet 302, the magnet 302, and thus the presence of the electrosurgical end effector 104, may be detected by the hall-effect sensor no matter the rotational position in which the end effector 104 is inserted.

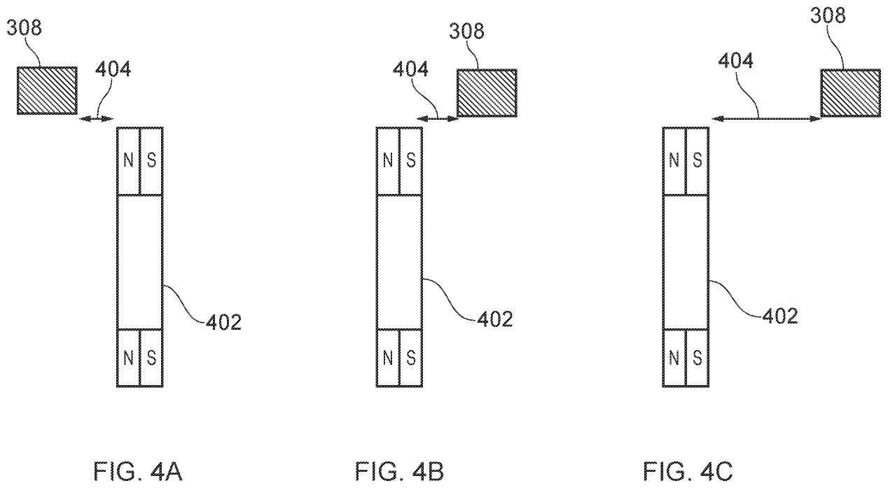

The ring-shape magnet may be a face-to-face polarised magnet. FIGS. 4a-4c show cut-away views of face-to-face polarised ring-shaped magnets 402 and hall-effect sensors 308. No other components are shown in these figures for ease of understanding, however it should be understood that the ring-shaped magnets 402 would be coupled to the end effector 104 of FIG. 2 and the hall effect sensor 308 would form part of the connecting portion 106 or handle portion 102 of the electrosurgical instrument 100.

The position of the magnet on the end-effector 104 may be such that, when coupled to the handle portion 102, the ring-shaped magnet 402 is offset compared to the hall effect sensor. For example, the ring-shaped magnet 402 may be offset in a direction along the rotational axis of the end effector 104, such that a position of the magnet 402 along the rotational axis of the end effector 104 identifies a function of the electrosurgical end effector 104. The ring-shaped magnet 402 may be mis-aligned or not aligned with the hall-effect sensor 308.

Different surgical instrument end effectors 104 may include the ring-shaped magnet 402 positioned in a different position on the end effector 104, such that the ring-shaped magnet 402 of different end effector types 104 includes a different offset 404 from the hall-effect sensor 308 when coupled to the handle portion 104. For example, FIGS. 4a-4c show face-to-face ring-shaped magnets 402 which have a different offset 404 from the hall-effect sensor 308. This may be achieved by positioning the magnet 402 in different positions on the end effector 104. Alternatively, or additionally, different strength magnets 402 which produce magnetic fields of different strength may be included on the end effectors 104 to indicate different types of end effector.

The hall-effect sensor 308 will detect the differences in the offset of the magnets 402 through the detection of the different magnetic fields produced.

For example, if north is facing the hall-effect sensor, as in FIG. 4a, the system may determine that end effector 104 is a burr instrument. If south is facing the hall-effect sensor 308, the system may determine that the end effector 104 is an RF shaver.

Further, the distance 404 between the magnet 402 and the hall-effect sensor 308 may also be used to determine different types of end effector 104, at least because the hall-effect sensor 308 will detect different magnetic fields for each position of the magnet 402 relative to the hall-effect sensor 308. Alternatively, different types of end effector may use magnets which have different magnetic field strengths, for example, stronger or weaker magnets which produce stronger or weaker magnetic fields.

The hall-effect sensor 308 may output a signal which is proportional to the strength of the detected magnetic field produced by the ring-shaped magnet 402. The strength of the field detected by the hall-effect sensor 308 may be dependent on the size/strength of the magnet and/or the distance of the magnet from the hall-effect sensor 308. The electrosurgical instrument 100 may include a processor configured to receive the output from the hall-effect sensor 308. Alternatively, the processor may be located externally to the electrosurgical instrument, for example in the wider electrosurgical system such as in an electrosurgical generator 202 or the suction source 204 to which the electrosurgical instrument 100 is coupled. The processor may determine the proximity of the ring-shaped magnet 402 and the hall-effect sensor 308 and any offset or distance between the hall-effect sensor 308 and the ring-shaped magnet 402. The processor may then determine the presence of the electrosurgical end effector 104 based on the presence of a magnetic field (and thus ring-shaped magnet 302). The processor may further determine the type of electrosurgical end effector 104 based on the magnitude or strength of the detected magnetic field. Based on this determination, signals provided to the electrosurgical instrument 100 from the electrosurgical generator 202 may be adapted depending on the type or function of the end effector 104 coupled to the electrosurgical instrument 100. For example, the generator 202 may provide an RF ablation signal if an RF electrosurgical end effector 104 is detected.

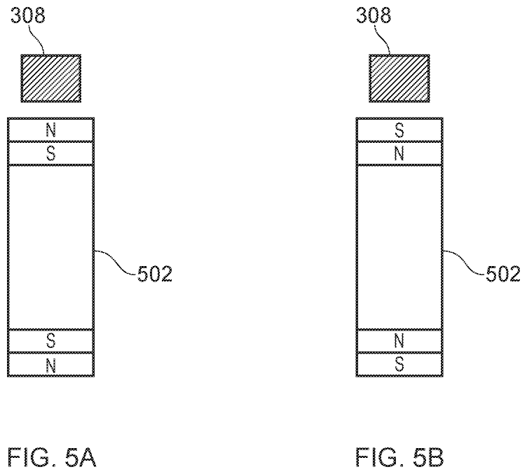

As shown in FIGS. 5a and 5b, the ring-shaped magnet may alternatively be a radially polarised magnet 502. The ring-shaped magnet 502 may be aligned with the hall-effect sensor 308 along a rotational axis of the electrosurgical end effector 104. The pole at the outer face of the magnet 502 may be north, as shown in FIG. 5a, or south, as shown in FIG. 5b. The processor included in the electrosurgical instrument 100 or wider electrosurgical system 200 may determine the type or function of the end effector 104 coupled to the electrosurgical instrument 100 based on the polarisation of the magnet 502 (for example, whether the north or south pole of the magnet is on the external edge of the magnet 502).

The electrosurgical end effector 104 may be an RF electrosurgical end effector, where the active edge 112 of the electrosurgical end effector 104 comprises an active electrode and a return electrode. The system may modify the RF signal applied to the electrosurgical end effector 104 depending on the type of RF electrosurgical end effector 104 which has been detected. The electrosurgical end effector 104 may be a rotary shaver end effector, and the active edge 112 of the electrosurgical end effector may comprise a rotary shaver arrangement.

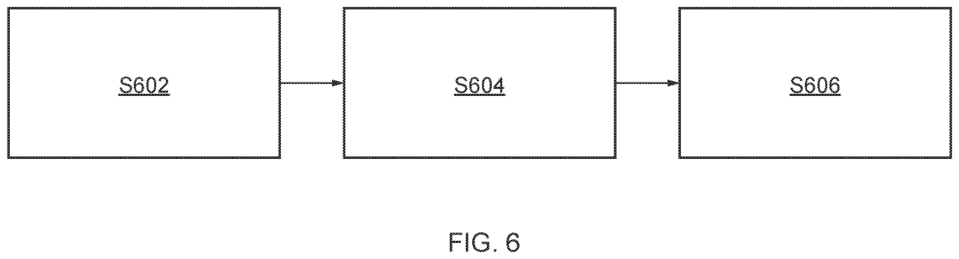

A method of determining the presence of the electrosurgical end effector and its coupling with a handle portion of the electrosurgical instrument is shown in FIG. 6. The system detects, in step S602, using a hall-effect sensor, the presence of a magnetic field produced by a ring-shaped magnet. An electrosurgical end effector of the electrosurgical instrument comprises the ring-shaped magnet, therefore detecting the presence of the ring-shaped magnet results in the detection of the electrosurgical end effector. Once the presence of an electrosurgical end effector has been determined, a function of the electrosurgical end effector may be determined in step S604 based on a magnitude of the detected magnetic field. Where the magnet is a face-to-face polarised magnet, determining the function of the electrosurgical end effector may comprise determining an offset between the ring-shaped magnet and the hall-effect sensor, for example, an offset between the ring-shaped magnet and the hall-effect sensor along a rotational axis of the electrosurgical end effector. Where the magnet is a radially polarised magnet, determining the function of the electrosurgical end effector may comprise determining the polarisation of the radially polarised magnet.

The function of the electrosurgical end effector may correspond to the type of electrosurgical end effector. For example, the electrosurgical end effector may be an RF electrosurgical end effector or a rotary shaver.

Once the function of the electrosurgical end effector has been determined, the electrosurgical system may supply, in step S606, control signals to the electrosurgical end effector based on the function of the electrosurgical end effector. For example, the system may supply an RF ablation signal when the function of the end effector is determined to be RF ablation. The control signals may be modified over time depending on the type of end effector.

Whilst the description presented herein refers primarily to an electrosurgical instrument, the description is also applicable to surgical instruments which are not electrosurgical. For example, the system may determine the function of a non-electrosurgical end effector.

Various modifications whether by way of addition, deletion, or substitution of features may be made to above described embodiment to provide further embodiments, any and all of which are intended to be encompassed by the appended claims.

1. An electrosurgical end effector for rotatably coupling to a surgical instrument handpiece, the surgical instrument handpiece comprising a hall effect sensor, the electrosurgical end effector comprising:
a distal end;
a proximal end comprising:
a ring-shaped magnet;
a connection means configured for rotatably coupling to the surgical instrument handpiece.

2. The electrosurgical end effector according to claim 1, wherein the ring-shaped magnet is a face-to-face polarised magnet.

3. The electrosurgical end effector according to claim 2, wherein a position of the magnet on the proximal end of the electrosurgical end effector identifies a function of the electrosurgical end effector.

4. The electrosurgical end effector according to claim 3, wherein the position of the magnet on the proximal end of the electrosurgical end effector is such that when the end effector is coupled to the surgical instrument handpiece, the relative position of the magnet and the hall effect sensor identifies the function of the electrosurgical end effector.

5. The electrosurgical end effector according to claim 3, wherein the position of the magnet along a rotational axis of the electrosurgical end effector identifies the function of the electrosurgical end effector.

6. The electrosurgical end effector according to claim 1, wherein the ring-shaped magnet is a radially polarised magnet.

7. The electrosurgical end effector according to claim 6, wherein the polarisation of the radially polarised magnet identifies a function of the electrosurgical end effector.

8. The electrosurgical end effector according to claim 1, wherein the electrosurgical end effector is an RF electrosurgical end effector, and the distal end of the electrosurgical end effector comprises an active electrode and a return electrode.

9. The electrosurgical end effector according to claim 1, wherein the electrosurgical end effector is a rotary shaver end effector, and the distal end of the electrosurgical end effector comprises a rotary shaver arrangement.

10. An electrosurgical instrument, the electrosurgical instrument comprising:
a handpiece; and
the electrosurgical end effector according to claim 1.

11. The electrosurgical instrument according to claim 10, wherein the handpiece comprises a hall-effect sensor.

12. The electrosurgical instrument according to claim 11, wherein the ring-shaped magnet of the electrosurgical end effector is aligned with the hall-effect sensor.

13. The electrosurgical instrument according to claim 11, wherein the ring-shaped magnet of the electrosurgical end effector is offset with respect to the hall-effect sensor along a rotational axis of the electrosurgical end effector.

14. The electrosurgical instrument according to claim 11, wherein the hall-effect sensor is configured to detect a magnetic field produced by the ring-shaped magnet.

15. The electrosurgical instrument according to claim 14, further comprising a processor configured to determine a function of the electrosurgical end effector based on an output of the hall-effect sensor.

16. A method of detecting the coupling of an electrosurgical end effector comprising a ring-shaped magnet to a surgical instrument handpiece, the method comprising:
detecting, using a hall-effect sensor, the presence of a magnetic field produced by the ring-shaped magnet.

17. The method according to claim 16, further comprising:
determining a function of the electrosurgical end effector based on a magnitude of the detected magnetic field.

18. The method according to claim 17, further comprising:
supplying a control signal to the electrosurgical end effector in dependence on the determined function of the electrosurgical end effector.

19. The method according to claim 17, wherein the ring-shaped magnet is a face-to-face polarised magnet, and wherein:
determining the function of the electrosurgical end effector further comprises determining an offset between the ring-shaped magnet and the hall-effect sensor along a rotational axis of the electrosurgical end effector.

20. The method according to claim 17, wherein the ring-shaped magnet is a radially polarised magnet, and wherein:
determining the function of the electrosurgical end effector further comprises determining the polarisation of the radially polarised magnet.
